(19) United States
(12) Patent Application Publication
(10) Pub. No.: US 2024/0307310 A1
(43) Pub. Date: Sep. 19, 2024

(54) STABILIZED SOLID ORAL PHARMACEUTICAL COMPOSITION OF VARENICLINE

(71) Applicant: LUPIN LIMITED, Mumbai, Maharashtra (IN)

(72) Inventors: Makarand Krishnakumar AVACHAT, Pune, Maharashtra (IN); Nikhil Prabhakar MALEWAR, Pune, Maharashtra (IN); Ramdoss Suresh KUMAR, Pune, Maharashtra (IN); Surendra Gangavishan PANPALIYA, Pune, Maharashtra (IN)

(73) Assignee: LUPIN LIMITED, Mumbai, Maharashtra (IN)

(21) Appl. No.: 18/681,334

(22) PCT Filed: Aug. 5, 2022
(86) PCT No.: PCT/IB2022/057305

§ 371 Date: Feb. 5, 2024

(30) Foreign Application Priority Data
Aug. 7, 2021 (IN) 202121035759

(51) Int. Cl. A61K 9/20; A61K 31/55

(52) U.S. Cl. CPC A61K 9/2009; A61K 9/2013; A61K 9/2054; A61K 31/55

(57) ABSTRACT

The disclosed invention relates to a stable pharmaceutical composition of varenicline or pharmaceutically acceptable salts thereof. More specifically the disclosed invention relates to a pharmaceutical composition comprising varenicline pharmaceutically acceptable salts thereof with daily acceptable limit of nitrosamine impurities.

### FIELD OF THE INVENTION

The present invention relates to stable pharmaceutical composition of varenicline or pharmaceutically acceptable salts thereof. In particular, the present invention provides a pharmaceutical composition comprising varenicline and pharmaceutically acceptable salts thereof with daily acceptable limit of nitrosamine impurities.

### BACKGROUND OF THE INVENTION

Nitrosamine describes a class of compounds having the chemical structure of a nitroso group bonded to an amine (R1N(-R2)—N═O). These compounds can form by a nitrosating reaction between amines (secondary, tertiary, or quaternary amines) and nitrous acid (nitrite salts under acidic conditions). Nitrosamine impurities are known to be mutagenic and carcinogenic.

In varenicline these impurities may be formed through reagent, catalyst, solvent or raw material. Also, these impurities may get formed when varenicline or salts thereof comes in contact with several excipients.

Recently many drug products have been recalled in US due to presence of nitrosamine impurities higher than acceptable intake (Al) limit. Some examples of such drug products are angiotensin II receptor blockers, ranitidine, nizatidine, and metformin.

In June 2018, FDA was informed of the presence of an impurity identified as N-nitrosodimethylamine (NDMA) in valsartan. In September 2019, FDA learned that some common heartburn products (ranitidine, commonly known as Zantac, and nizatidine, commonly known as Axid) contained unacceptable levels of NDMA. In April 2020, FDA requested that all ranitidine products be withdrawn from the US market. In June 2021, Pfizer suspended worldwide distribution of Chantix® (varenicline tartrate tablets) after finding unacceptable levels of nitrosamines in certain Chantix® lots.

Nitrosamine impurities may be introduced in finished products through active pharmaceutical ingredients (APIs) or excipients being used. Nitrosamine impurities can be formed by a nitrosating reaction between amines and nitrous acid (nitrite salts under acidic condition). Hence if the API is kept in the vicinity of alkaline or basic excipients, formation of nitrosamine impurity can be avoided.

Varenicline is a nicotinic receptor partial agonist and binds with high affinity and selectivity at α4β2 neuronal nicotinic acetylcholine receptors. It is indicated for use as an aid to smoking cessation treatment. It blocks the ability of nicotine to activate α4β2 receptors and thus to stimulate the central nervous mesolimbic dopamine system, which is responsible for its role in smoking cessation. It both reduces craving for and decreases the pleasurable effects of nicotine from cigarettes and other tobacco products.

Varenicline is approved with the brand names Chantix®/Champix®, immediate release tablets with varenicline tartrate doses of 1 and 2 mg free base, to be administered orally. However, due to recent recall of Chantix® batches from market, it is currently in shortage worldwide especially in US and Europe.

Due to current shortage of Chantix®, patients would have to switch to alternative therapy such as nicotine replacement therapy and bupropion. Recently, USFDA temporarily allowed distribution of varenicline tablets with elevated levels of impurities, to maintain availability of the anti-smoking tablets, as the health benefits of stopping smoking outweigh the cancer risk from the nitrosamine impurity in varenicline.

Thus, there is an unmet need to provide an alternate dosage form of varenicline which has n-nitrosamine impurity within the acceptable limit. Surprisingly, it was found that using basic or alkaline excipients in dosage form of varenicline, has reduced the nitrosamine impurity. Alternatively, varenicline composition with organic or inorganic acids selected from adipic acid, fumaric acid, citric acid or tartaric acid also reduced the amount of nitrosamine impurity within the acceptable limit.

### SUMMARY OF THE INVENTION

It is therefore among the objects of the present invention to provide in certain of its preferred aspects and preferred embodiments each and all the following.

The present invention, accordingly, provides a stable composition comprising varenicline or pharmaceutically acceptable salts thereof, wherein the composition contains nitrosamine impurities within daily acceptable limit.

In a further embodiment the present invention, provides a stable composition comprising varenicline or pharmaceutically acceptable salts thereof, wherein the composition contains nitrosamine impurities within daily acceptable limit and wherein the daily acceptable limit of nitrosamine impurity is 9 ppm.

In an embodiment the invention provides a stable composition comprising varenicline or pharmaceutically acceptable salts thereof, wherein the composition contains nitrosamine impurities within daily acceptable limit, and wherein the daily acceptable limit of nitrosamine impurity is 9 ppm and wherein nitrosamine impurities include but are not limited to N-nitroso varenicline and N-nitrosodiethylamine.

In yet another embodiment the invention provides a stable composition comprising varenicline or pharmaceutically acceptable salts thereof and alkaline or basic excipients with or without other pharmaceutically acceptable excipients.

A further embodiment of the invention is to provide a stable composition comprising varenicline or pharmaceutically acceptable salts thereof and alkaline or basic excipients, wherein the composition contains nitrosamine impurities within daily acceptable limit.

A further embodiment of the invention is to provide a stable composition comprising varenicline or pharmaceutically acceptable salts thereof and alkaline or basic excipients, wherein the composition contains nitrosamine impurities within daily acceptable limit and wherein the daily acceptable limit of nitrosamine impurity is 9 ppm.

An embodiment of the invention is to provide a stable composition comprising varenicline or pharmaceutically acceptable salts thereof and alkaline or basic excipients, wherein the composition contains nitrosamine impurities within daily acceptable limit, wherein the daily acceptable limit of nitrosamine impurity is 9 ppm and wherein nitrosamine impurities include but are not limited to N-nitroso varenicline and N-nitrosodiethylamine.

In a further embodiment the invention provides a stable composition comprising varenicline or pharmaceutically acceptable salts thereof and alkaline or basic excipients, wherein alkaline or basic excipients are either organic or inorganic by nature or a combination of both.

Yet another embodiment of the invention is to provide a stable composition comprising varenicline or pharmaceutically acceptable salts thereof and alkaline or basic excipients, wherein alkaline or basic excipients are either organic or inorganic by nature or a combination of both and wherein the composition contains nitrosamine impurities within daily acceptable limit.

In another embodiment of the invention is to provide a stable composition comprising varenicline or pharmaceutically acceptable salts thereof and alkaline or basic excipients, wherein alkaline or basic excipients are either organic or inorganic by nature or a combination of both and wherein the composition contains nitrosamine impurities within daily acceptable limit and wherein the daily acceptable limit of nitrosamine impurity is 9 ppm.

In another embodiment of the invention is to provide a stable composition comprising varenicline or pharmaceutically acceptable salts thereof and alkaline or basic excipients, wherein alkaline or basic excipients are either organic or inorganic by nature or a combination of both and wherein the composition contains nitrosamine impurities within daily acceptable limit, wherein the daily acceptable limit of nitrosamine impurity is 9 ppm and wherein nitrosamine impurities include but are not limited to N-nitroso varenicline and N-nitrosodiethylamine.

In yet another embodiment the invention provides a stable composition comprising varenicline or pharmaceutically acceptable salts thereof and alkaline or basic excipients wherein the organic alkaline or basic excipients include but are not limited to carbohydrates, meglumine, starch, cellulose, petrochemicals, povidones, mineral hydrocarbons, acrylic polymers, oleochemicals, proteins or combinations thereof.

In yet another embodiment the invention provides a stable composition comprising varenicline or pharmaceutically acceptable salts thereof and alkaline or basic excipients wherein the organic alkaline or basic excipients include but are not limited to carbohydrates, meglumine, starch, cellulose, petrochemicals, povidones, mineral hydrocarbons, acrylic polymers, oleochemicals, proteins or combinations thereof and wherein the composition contains nitrosamine impurities within daily acceptable limit.

In yet another embodiment the invention provides a stable composition comprising varenicline or pharmaceutically acceptable salts thereof and alkaline or basic excipients wherein the organic alkaline or basic excipients include but are not limited to carbohydrates, meglumine, starch, cellulose, petrochemicals, povidones, mineral hydrocarbons, acrylic polymers, oleochemicals, proteins or combinations thereof and wherein the composition contains nitrosamine impurities within daily acceptable limit and wherein the daily acceptable limit of nitrosamine impurity is 9 ppm.

In yet another embodiment the invention provides a stable composition comprising varenicline or pharmaceutically acceptable salts thereof and alkaline or basic excipients wherein the organic alkaline or basic excipients include but are not limited to carbohydrates, meglumine, starch, cellulose, petrochemicals, povidones, mineral hydrocarbons, acrylic polymers, oleochemicals, proteins or combinations thereof and wherein the composition contains nitrosamine impurities within daily acceptable limit, wherein the daily acceptable limit of nitrosamine impurity is 9 ppm and wherein nitrosamine impurities include but are not limited to N-nitroso varenicline and N-nitrosodiethylamine.

In yet another embodiment the invention provides a stable composition comprising varenicline or pharmaceutically acceptable salts thereof and alkaline or basic excipients wherein the inorganic alkaline or basic excipients include but are not limited to metal or non-metal phosphates, carbonates, sulfate, halites, stearates, oxides, silica or combinations thereof.

In yet another embodiment the invention provides a stable composition comprising varenicline or pharmaceutically acceptable salts thereof and alkaline or basic excipients wherein the inorganic alkaline or basic excipients include but are not limited to metal or non-metal phosphates, carbonates, bicarbonates, sulfate, halites, stearates, oxides, silica or combinations thereof and wherein the composition contains nitrosamine impurities within daily acceptable limit.

In yet another embodiment the invention provides a stable composition comprising varenicline or pharmaceutically acceptable salts thereof and alkaline or basic excipients wherein the inorganic alkaline or basic excipients include but are not limited to metal or non-metal phosphates, carbonates, bicarbonates, sulfate, halites, stearates, oxides, silica or combinations thereof and wherein the composition contains nitrosamine impurities within daily acceptable limit and wherein the daily acceptable limit of nitrosamine impurity is 9 ppm.

In another embodiment the invention provides a stable composition comprising varenicline or pharmaceutically acceptable salts thereof and alkaline or basic excipients wherein the inorganic alkaline or basic excipients include but are not limited to metal or non-metal phosphates, carbonates, bicarbonates, sulfate, halites, stearates, oxides, silica or combinations thereof and wherein the composition contains nitrosamine impurities within daily acceptable limit, wherein the daily acceptable limit of nitrosamine impurity is 9 ppm and wherein nitrosamine impurities include but are not limited to N-nitroso varenicline and N-nitrosodiethylamine.

In another embodiment the invention provides a stable composition comprising varenicline or pharmaceutically acceptable salts thereof and one or more pharmaceutically acceptable excipients, wherein weight percent of varenicline base is 0.4 to 1.2% or more, preferably 0.7% or 0.8%, more preferably 0.75% as that of total weight of composition.

In another embodiment the invention provides a stable composition comprising varenicline or pharmaceutically acceptable salts thereof and one or more pharmaceutically acceptable excipients, wherein weight percent of varenicline base is 0.4 to 1.2% or more, preferably 0.7% or 0.8%, more preferably 0.75% as that of total weight of composition, wherein the composition contains nitrosamine impurities within daily acceptable limit.

In another embodiment the invention provides a stable composition comprising varenicline or pharmaceutically acceptable salts thereof and one or more pharmaceutically acceptable excipients, wherein weight percent of varenicline base is 0.4 to 1.2% or more, preferably 0.7% or 0.8%, more preferably 0.75% as that of total weight of composition, wherein the composition contains nitrosamine impurities within daily acceptable limit and wherein the daily acceptable limit of nitrosamine impurity is 9 ppm.

In another embodiment the invention provides a stable composition comprising varenicline or pharmaceutically acceptable salts thereof and one or more pharmaceutically acceptable excipients, wherein weight percent of varenicline base is 0.4 to 1.2% or more, preferably 0.7% or 0.8%, more preferably 0.75% as that of total weight of composition, wherein the composition contains nitrosamine impurities within daily acceptable limit, wherein the daily acceptable limit of nitrosamine impurity is 9 ppm and wherein the nitrosamine impurity is N-nitroso varenicline and N-nitrosodiethylamine.

In another embodiment the invention provides a stable composition comprising varenicline or pharmaceutically acceptable salts thereof and organic acid or inorganic acids.

In yet another embodiment the invention provides a stable composition comprising varenicline or pharmaceutically acceptable salts thereof and organic acid or inorganic acids, wherein the composition contains nitrosamine impurities within daily acceptable limit.

In yet another embodiment the invention provides a stable composition comprising varenicline or pharmaceutically acceptable salts thereof and organic acid or inorganic acids, wherein the composition contains nitrosamine impurities within daily acceptable limit and wherein the daily acceptable limit of nitrosamine impurity is 9 ppm.

In yet another embodiment the invention provides a stable composition comprising varenicline or pharmaceutically acceptable salts thereof and organic acid or inorganic acids, wherein the composition contains nitrosamine impurities within daily acceptable limit, wherein the daily acceptable limit of nitrosamine impurity is 9 ppm and wherein the nitrosamine impurity is N-nitroso varenicline and N-nitrosodiethylamine.

In another embodiment the invention provides a stable composition comprising varenicline or pharmaceutically acceptable salts thereof and organic acid or inorganic acids wherein the organic acid or inorganic acids include but are not limited to tartaric, fumaric, citric or adipic acid.

In yet another embodiment the invention provides a stable composition comprising varenicline or pharmaceutically acceptable salts thereof and organic acid or inorganic acids wherein the organic acid or inorganic acids include but are not limited to tartaric, fumaric, citric or adipic acid and wherein the composition contains nitrosamine impurities within daily acceptable limit.

In yet another embodiment the invention provides a stable composition comprising varenicline or pharmaceutically acceptable salts thereof and organic acid or inorganic acids wherein the organic acid or inorganic acids include but are not limited to tartaric, fumaric, citric or adipic acid, wherein the composition contains nitrosamine impurities within daily acceptable limit and wherein the daily acceptable limit of nitrosamine impurity is 9 ppm.

In yet another embodiment the invention provides a stable composition comprising varenicline or pharmaceutically acceptable salts thereof and organic acid or inorganic acids, wherein the organic acid or inorganic acids include but are not limited to tartaric, fumaric, citric or adipic acid, wherein the composition contains nitrosamine impurities within daily acceptable limit, wherein the daily acceptable limit of nitrosamine impurity is 9 ppm and wherein the nitrosamine impurity is N-nitroso varenicline and N-nitrosodiethylamine.

The details of one or more embodiments of the invention set forth below are illustrative only and not intended to limit to the scope of the invention. Other features, objects and advantages of the inventions will be apparent from the description and claims.

### DETAIL DESCRIPTION OF INVENTION

Nitrosamine impurities are known to be mutagenic and carcinogenic, very small exposure of these impurities can lead to cancer.

The terms “nitrosamine” and “N-nitrosamine” are used interchangeably and should both be understood to refer to the following structure:

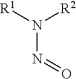

USFDA has identified seven nitrosamine impurities that theoretically could be present in drug products: N-nitrosodimethylamine (NDMA), N-nitrosodiethylamine (NDEA), N-nitroso-N-methyl-4-aminobutanoic acid (NMBA), N-nitrosoisopropylethyl amine (NIPEA), N-nitrosodiisopropylamine (NDIPA), N-nitrosodibutylamine (NDBA), and N-nitrosomethylphenylamine (NMPA). Five of them (NDMA, NDEA, NMBA, NIPEA, and NMPA) have actually been detected in drug substances or drug products.

Chemical structures of seven potential nitrosamine impurities in active pharmaceutical ingredients and drug products are given below:

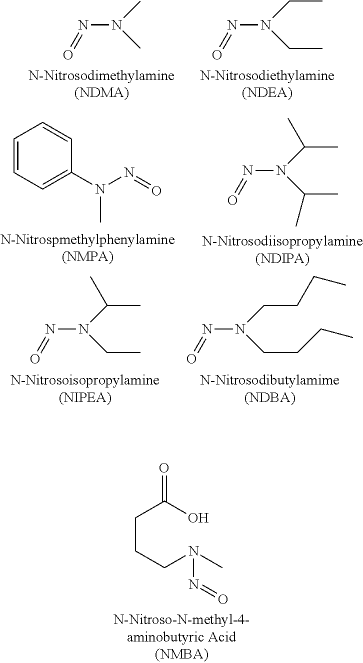

The ICH guidance recommends control of any known mutagenic carcinogen, such as nitroso-compounds, at or below a level such that there would be a negligible human cancer risk associated with the exposure to potentially mutagenic impurities. Following the discovery of nitrosamine contaminants in angiotensin receptor blockers (ARBs), FDA published interim acceptable limits for these impurities. FDA recommended that manufacturers take action to quantify nitrosamine levels in their drugs and to reduce or remove these impurities when above the interim limit; FDA has used the interim limits to guide immediate decision-making for additional evaluation and product recalls while balancing the risks of potential long-term carcinogen exposure with disruption to clinical management of patients.

The recent guidance of FDA published in September 2020 (Control of nitrosamine impurities in human drugs), provides several route causes for the presence of nitrosamine impurities in APIs. However, even if the APIs have acceptable limit of nitrosamine impurities, once incorporated in a formulation along with excipients, there are chances of formation of nitrosamine impurities due to interaction of different chemical compounds.

FDA recommends the following acceptable intake (Al) limits fir the nitrosamine impurities a s given in the below table:

TABLE 1
AI Limits for NDMA, NDEA, NMBA, NMPA,
NIPEA, and NDIPA in Drug Products

| Nitrosamine | AI Limit (ng/day) [symbol] |
|---|---|
| NDMA | 96 |
| NDEA | 26.5 |
| NMBA | 96 |
| NMPA | 26.5 |
| NIPEA | 26.5 |
| NDIPA | 26.5 |

[symbol]

indicates data missing or illegible when filed

The conversion of Al limit into ppm varies by product and is calculated based on a drug's maximum daily dose (MDD) as reflected in the drug label (ppm=Al (ng)/MDD(mg)).

These limits are applicable only if a drug product contains a single nitrosamine. If more than one of the nitrosamine impurities identified in Table 1 is detected and the total quantity of nitrosamine impurities exceeds 26.5 ng/day (the Al for the most potent nitrosamines) based on the maximum daily dose (MDD), the manufacturer should contact the Agency for evaluation.

Varenicline is a nicotinic receptor partial agonist. Specifically, varenicline is partial agonist of the alpha4/beta2 subtype of the nicotinic acetylcholine receptor. Varenicline is approved as immediate release tablet containing varenicline tartrate salt, with brand names Chantix® or Champix® in US and Europe respectively. It is indicated for use as an aid to smoking cessation treatment. Chantix® is approved with two doses equivalent to 0.5 mg and 1 mg.

On Jun. 25, 2021 Pfizer suspended global distribution of Chantix® after finding unacceptable limit of nitrosamine impurities in some of the batches.

Apart from NDMA there is another nitrosamine impurity, N-nitroso-varenicline which is usually found in APIs as well as finished product of varenicline. As per the recent update, USFDA has provided acceptable intake limit of N-nitroso-varenicline (VC09) from 37 ng/day to 185 ng/day, in finished product of varenicline.

Additionally, European Medicines Agency (EMA) has also provided the daily acceptable intake limit for N-nitroso-varenicline impurity as 18 ng/day. In view of the above limit minimum daily intake limit for VC09 was determined as 9 ppm.

Varenicline means varenicline or pharmaceutically acceptable salts thereof.

Salts of varenicline may include but not limited to tartrate, hydrochloride, sulfate, mesylate, fumarate. Most preferable salt of varenicline is tartrate.

Alkaline or basic excipient(s) used herein, are either organic or inorganic by nature or a combination of both.

Organic alkaline or basic excipients include but are not limited to carbohydrates, meglumine, starch, cellulose, petrochemicals, povidones, mineral hydrocarbons, acrylic polymers, oleochemicals, amines or proteins or combinations thereof.

Inorganic alkaline or basic excipients include but are not limited to metal or non-metal phosphates, carbonates, sulfate, halites, stearates, oxides, silica or combinations thereof, preferably dicalcium phosphate, sodium carbonate, sodium bicarbonate and colloidal silicon dioxide.

An aspect of the invention is a stable pharmaceutical composition comprising varenicline or pharmaceutically acceptable salts thereof comprising alkaline or basic excipient with or without other pharmaceutically acceptable excipients, wherein the composition contains nitrosamine impurities within daily acceptable limit and wherein the nitrosamine impurities are N-nitroso varenicline and N-nitrosodiethylamine.

Another aspect of the invention is a stable pharmaceutical composition of varenicline or pharmaceutically acceptable salts thereof comprising alkaline or basic excipient with or without other pharmaceutically acceptable excipients, wherein weight ratio of varenicline to alkaline or basic excipient is about 1:2 to 1:90.

Yet another aspect of the invention is a stable pharmaceutical composition comprising varenicline or pharmaceutically acceptable salts thereof comprising alkaline or basic excipient with or without other pharmaceutically acceptable excipients, wherein weight ratio of varenicline to alkaline or basic excipient is about 1:2 to 1:90 and wherein the composition contains nitrosamine impurities within daily acceptable limit and wherein the nitrosamine impurities are N-nitroso varenicline and N-nitrosodiethylamine.

An aspect of the invention is a stable pharmaceutical composition of varenicline or pharmaceutically acceptable salts thereof comprising dicalcium phosphate and pharmaceutically acceptable excipients.

An aspect of the invention is a stable pharmaceutical composition of varenicline or pharmaceutically acceptable salts thereof comprising sodium bicarbonate and pharmaceutically acceptable excipients.

An aspect of the invention is a stable pharmaceutical composition of varenicline or pharmaceutically acceptable salts thereof comprising dicalcium phosphate, sodium bicarbonate and pharmaceutically acceptable excipients.

Yet another aspect of the invention is a stable pharmaceutical composition comprising varenicline or pharmaceutically acceptable salts thereof comprising dicalcium phosphate and/or sodium bicarbonate with or without other pharmaceutically acceptable excipients, wherein the composition contains nitrosamine impurities within daily acceptable limit and wherein the nitrosamine impurities are N-nitroso varenicline and N-nitrosodiethylamine.

An aspect of the invention is a stable pharmaceutical composition of varenicline or pharmaceutically acceptable salts thereof and comprising pharmaceutically acceptable excipients, wherein weight percent of varenicline base is 0.70% or more as that of total weight of composition.

Another aspect of the invention is a stable pharmaceutical composition of varenicline or pharmaceutically acceptable salts thereof and comprising pharmaceutically acceptable excipients, wherein weight percent of varenicline base is 0.70% or more as that of total weight of composition and wherein the composition contains nitrosamine impurities within daily acceptable limit.

Yet another aspect of the invention is a stable pharmaceutical composition comprising varenicline or pharmaceutically acceptable salts thereof and comprising pharmaceutically acceptable excipients, wherein weight percent of varenicline base is 0.70% or more as that of total weight of composition, wherein the composition contains nitrosamine impurities within daily acceptable limit and wherein the nitrosamine impurities are N-nitroso varenicline and N-nitrosodiethylamine

Another aspect of the invention provides a stable composition of varenicline or pharmaceutically acceptable salts thereof comprising tartaric acid and other pharmaceutically acceptable excipients.

Another aspect of the invention provides a stable composition of varenicline or pharmaceutically acceptable salts thereof comprising tartaric acid and other pharmaceutically acceptable excipients, wherein weight percent of varenicline base is 0.7% or more as that of total weight of composition.

Another aspect of the invention provides a stable composition of varenicline or pharmaceutically acceptable salts thereof comprising tartaric acid and pharmaceutically acceptable excipients, wherein weight ratio of varenicline and tartaric acid is 0.008:1 to 0.025:1.

An aspect of the invention is a stable pharmaceutical composition comprising varenicline or pharmaceutically acceptable salts thereof and tartaric acid with or without other pharmaceutically acceptable excipients, wherein the composition contains nitrosamine impurities within daily acceptable limit and wherein the nitrosamine impurities are N-nitroso varenicline and N-nitrosodiethylamine.

As described herein, the daily acceptable limit of nitrosamine impurity is 9 ppm.

Depending upon the manner of introduction, the compounds described herein may be formulated in a variety of ways. The term pharmaceutical composition/formulation/dosage herein comprises various form as used pharmaceutically acceptable dosage forms including oral solid as well as liquid dosage forms, such as but not limited to, tablets, soft gelatin capsule, capsules (filled with powders, powders for reconstitution, pellets, beads, mini-tablets, pills, micro-pellets, small tablet units, MUPS, disintegrating tablets, dispersible tablets, granules, sprinkles microspheres and multi-particulates), sachets (filled with powders, pellets, beads, mini-tablets, pills, micro-pellets, small tablet units, MUPS, disintegrating tablets, dispersible tablets, granules, sprinkles microspheres and multi-particulates) and sprinkles, liquids, liquid dispersions, suspensions, solutions, emulsions, sprays, spot-on) and the like; parenteral dosage forms such as liquids, liquid dispersions, suspensions, solutions, emulsions, (rods, capsules, rings), lyophilized formulation for reconstitution or freeze-dried powder, ready-to-use-liquid and the like; topical dosage forms, such as but not limited to, sprays, solutions, suspensions, ointments, drops, in-situ gel, aerosols, ointments, microspheres, creams, gels, patches, films etc.

Pharmaceutically acceptable excipients refer to non-API or inactive substances which may be selected, for example, from adjuvants, carriers, binders, lubricants, disintegrants, diluents, glidants, stabilising agents, buffering agents, emulsifying agents, viscosity-regulating agents, surfactants, preservatives, flavourings and colorants.

Suitable diluents include substances such as lactose, starch and pregelatinized starch, sucrose, mannitol, sorbitol, powdered and microcrystalline cellulose, calcium phosphates or combinations thereof.

Suitable binders include substances such as celluloses (e.g., cellulose, methylcellulose, ethylcellulose, hydroxypropyl cellulose and hydroxymethylcellulose), polypropylpyrrolidone, polyvinylprrolidone, gelatin, gum arabic, polyethylene glycol, starch, natural and synthetic gums (e.g., acacia, alginates, and gum arabic) and waxes.

Suitable lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc and zinc stearate. A preferred lubricant is magnesium stearate.

Suitable disintegrants include sodium starch glycolate, sodium carboxymethyl cellulose, calcium carboxymethyl cellulose, croscarmellose sodium, polyvinylpyrrolidone, methyl cellulose, microcrystalline cellulose, powdered cellulose, lower alkyl-substituted hydroxypropyl cellulose, polacrilin potassium, starch, pregelatinized starch and sodium alginate.

Examples of glidants include silicon dioxide, talc and cornstarch.

Process for preparing composition of the present invention comprises but are not limited to wet granulation, dry granulation, direct compression, hot melt extrusion, spray drying process and solid dispersion.

Process for preparing composition of the present invention further comprises blending the API with excipients by blender mixer or by turbo rapid variable mixer wherein blender mixer is preferable.

The pharmaceutical formulations may be used for both medical therapeutic (acute or chronic) and/or prophylactic (prevention) administration as appropriate. The dose, frequency and duration will vary depending on such factors as the nature and severity of the condition being treated, the age and general health of the host and the tolerance of the host to the active ingredient. The pharmaceutical composition or medicament may be given in a single daily dose, in multiple doses during the day or even in a weekly dose. The regimen may last from about 2-3 days to several weeks or longer. Typically, the composition is administered to a human patient once or twice a day with a unit dosage of about 0.25 mg to about 10.0 mg, but the above dosage may be properly varied depending on the age, body weight and medical condition of the patient and the type of administration.

The following examples are illustrative of the present invention, and the examples should not be considered as limiting the scope of this invention in any way, as these examples and other equivalents thereof will become apparent to those versed in the art, in the light of the present disclosure, and the claims.

### EXAMPLES

### Formulation Details with Basic or Alkaline Excipients

| Ingredient | % w/w Range | | | |
|---|---|---|---|---|
| Varenicline Tartrate | More than 0.7% | | | |
| (Varenicline) | | | | |
| Dibasic Calcium | 0.25-98% | 0.25-98% | 0.25-98% | 0.25-98% |
| Phosphate | | | | |
| Magnesium Stearate | 0.5-5% | 0.5-5% | 0.5-5% | 0.5-5% |
| Mannitol | — | 2.00-99.75% | — | — |
| Sodium Carbonate | — | — | 0.25-98% | — |
| or Bicarbonate | | | | |
| Croscarmellose | — | — | — | 0.1-5.0% |
| sodium | | | | |

### Procedure

1. These cores can be prepared by direct compression, wet granulation (with a high or low shear wet granulator or fluid bed granulator), extrusion granulation, rotogranulation or roller compaction.
2. The tablets of the invention may further comprise a film coating.

Formulation 1:

| Ingredient | mg/tab | % w/w |
|---|---|---|
| Varenicline Tartrate (Varenicline) | 1.71 (1.00) | 1.43 |
| Dibasic Calcium Phosphate | 117.040 | 97.53 |
| Magnesium Stearate | 1.25 | 1.04 |
| Core tablets | 120.00 | 100.00 |

### Procedure

1. Sift Varenicline Tartrate and Dibasic Calcium Phosphate through 25 #S.S. sieve and mix.
2. Sift Magnesium Stearate through 40 #S.S. sieve and mix with drug-mix of step 1.
3. Compress into tablets using suitable tooling.

Formulation 2:

| Ingredient | mg/tab | % w/w |
|---|---|---|
| Varenicline Tartrate (Varenicline) | 1.71 (1.00) | 1.43 |
| Mannitol | 92.04 | 76.70 |
| Dibasic Calcium Phosphate | 25.00 | 20.83 |
| Magnesium Stearate | 1.25 | 1.04 |
| Core tablets | 120.00 | 100.00 |

### Procedure

1. Sift Varenicline Tartrate and Dibasic Calcium Phosphate through 25 #S.S. Sieve and mix.
2. Sift Mannitol through 25 #S.S. sieve and mix with drug-mix of step 1.
3. Sift Magnesium Stearate through 40 #S.S. sieve and mix with drug-mix of step 2.
4. Compress into tablets using suitable tooling.

Formulation 3:

| Ingredient | mg/tab | % w/w |
|---|---|---|
| Varenicline Tartrate (Varenicline) | 1.71 (1.00) | 1.43 |
| Dibasic Calcium Phosphate | 92.04 | 76.70 |
| Sodium Bicarbonate | 25.00 | 20.83 |
| Magnesium Stearate | 1.25 | 1.04 |
| Core tablets | 120.00 | 100.00 |

### Procedure

1. Sift Varenicline Tartrate and Dibasic Calcium Phosphate through 25 #S.S. Sieve and mix.
2. Sift Sodium Bicarbonate through 25 #S.S. sieve and mix with drug-mix of step 1.
3. Sift Magnesium Stearate through 40 #S.S. sieve and mix with drug-mix of step 2.
4. Compress into tablets using suitable tooling.

Formulation 4:

| Ingredient | mg/tab | % w/w |
|---|---|---|
| Varenicline Tartrate (Varenicline) | 0.855 (0.500) | 1.43 |
| Dibasic Calcium Phosphate | 58.145 | 96.91 |
| Croscarmellose Sodium | 0.500 | 0.83 |
| Magnesium Stearate | 0.500 | 0.83 |
| Core tablets | 60.000 | 100.00 |

### Procedure

1. Sift Varenicline Tartrate and Dibasic Calcium Phosphate through 25 #S.S. Sieve and mix.
2. Sift Croscarmellose Sodium through 25 #S.S. sieve and mix with drug-mix of step 1.
3. Sift Magnesium Stearate through 40 #S.S. sieve and mix with drug-mix of step 2.
4. Compress into tablets using suitable tooling.

Formulation Details with Weak Acids:

| Ingredient | % w/w Range | |
|---|---|---|
| Varenicline Tartrate (Varenicline) | More than 0.7% | |
| Tartaric acid | 0.25-98% | 0.25-98% |
| Magnesium Stearate | 0.5-5% | 0.5-5% |
| Mannitol | — | 2.00-99.75% |

### Procedure

1. These cores can be prepared by direct compression, wet granulation (with a high or low shear wet granulator or fluid bed granulator), extrusion granulation, rotogranulation or roller compaction.
2. The tablets of the invention may further comprise a film coating.

Formulation 5:

| Ingredient | mg/tab | % w/w |
|---|---|---|
| Varenicline Tartrate (Varenicline) | 1.71 (1.00) | 1.43 |
| Tartaric Acid | 117.040 | 97.53 |
| Magnesium Stearate | 1.25 | 1.04 |
| Core tablets | 120.00 | 100.00 |

### Procedure

1. Sift Varenicline Tartrate and Tartaric Acid through 25 #S.S. Sieve and mix.
2. Sift Magnesium Stearate through 40 #S.S. sieve and mix with drug-mix of step 1.
3. Compress into tablets using suitable tooling.

Formulation 6:

| Ingredient | mg/tab | % w/w |
|---|---|---|
| Varenicline Tartrate (Varenicline) | 1.71 (1.00) | 1.43 |
| Tartaric Acid | 80.00 | 66.67 |
| Mannitol | 37.04 | 30.87 |
| Magnesium Stearate | 1.25 | 1.04 |
| Core tablets | 120.00 | 100.00 |

### Procedure

1. Sift Varenicline Tartrate and Tartaric Acid through 25 #S.S. Sieve and mix.
2. Sift Mannitol through 25 #S.S. sieve and mix with drug-mix of step 1.
3. Sift Magnesium Stearate through 40 #S.S. sieve and mix with drug-mix of step 2.
4. Compress into tablets using suitable tooling.

Formulation 7 and 8:

| | FORMULATION 7 | | FORMULATION 8 | |
|---|---|---|---|---|
| Ingredients | 0.5 mg | 1.0 mg | 0.5 mg | 1.0 mg |
| Varenicline Tartrate | 0.855 | 1.710 | 0.855 | 1.710 |
| eq. to Varenicline | (0.500) | (1.000) | (0.500) | (1.000) |
| Microcrystalline | 58.145 | 116.290 | 61.895 | 123.790 |
| Cellulose | | | | |
| Anhydrous Dibasic | 33.500 | 67.000 | 33.500 | 67.000 |
| Calcium Phosphate | | | | |
| Povidone | 4.500 | 9.000 | — | — |
| Purified Water # | Quantity | Quantity | — | — |
| | sufficient | sufficient | | |
| Extragranular | | | | |
| Croscarmellose | 2.000 | 4.000 | 2.000 | 4.000 |
| Sodium | | | | |
| Colloidal Silicon | 0.500 | 1.000 | 0.500 | 1.000 |
| Dioxide | | | | |
| Magnesium Stearate | 0.500 | 1.000 | 1.250 | 2.500 |
| Core tablet weight | 100.000 | 200.000 | 100.000 | 200.000 |
| Coating | | | | |
| Opadry White | 3.000 | 6.000 | 3.000 | 6.000 |
| 03B28796/ | (Opadry | (Opadry | (Opadry | (Opadry |
| Opadry Blue | white) | blue) | white) | blue) |
| 03B90570 | | | | |
| Purified Water | Quantity | Quantity | Quantity | Quantity |
| USP # | sufficient | sufficient | sufficient | sufficient |
| Coated tablet weight | 103.000 | 206.000 | 103.000 | 206.000 |

### Analysis of Nitrosamine Impurities

TABLE 2

| | Average | | |
|---|---|---|---|
| | tablet | Nitrosamines | |
| Material Description and | weight | Results in ppm | |
| process adopted | (mg) | VC09 | NDEA |
| Drug Substance (Initial) | NA | 0.23 | ND |
| Drug Substance (40/75, 7 days, open) | NA | 0.20 | ND |
| Drug Substance (60° C, 7 days, open) | NA | 0.27 | ND |
| Chantix ® Tablets 1 mg | 204.54 | 120.05 | ND |
| Chantix ® Tablets 0.5 mg | 106.11 | 421.57 | ND |
| Formulation 7, 1.0 mg (Initial) | 206.00 | 39.14 | ND |
| Formulation 7, 0.5 mg (Initial) | 103 | 43.63 | ND |

Although the amounts of nitrosamine are not less than 9 ppm, it was far less as compared to Chantix® tablets. Further, FDA has temporarily allowed some manufacturers to distribute varenicline containing impurities above its intake limit of 37 nanograms per day, but below an interim limit of 185 ng per day, until the impurity can be eliminated or reduced to acceptable levels. Levels of nitrosamine in Formulation 7 are significantly lower than interim limit allowed by FDA.

TABLE 3

| | Aver- | | |
|---|---|---|---|
| | age | | |
| | weight | | |
| | of | Nitrosamines | |
| | tablet | in ppm | |
| Batch details | (mg) | VC09 | NDEA |

Batch with Tartaric Acid-1—Formulation 5

| | | | |
|---|---|---|---|
| Dry Mix 15 Min-1 mg-Initial | 118.75 | 0.19 | ND |
| Lubricated Blend-1 mg-Initial | 120 | 0.17 | ND |
| Core Tablets-1 mg-Initial | 120 | 0.18 | ND |
| Core Tablets-1 mg-Initial | 120 | 0.15 | ND |
| (40/75, 7 days, Open) | | | |
| Core Tablets-1 mg-Initial | 120 | 0.43 | ND |
| (60°, 7 days, Open) | | | |

Batch with Tartaric Acid-2—Formulation 6

| | | | |
|---|---|---|---|
| Dry Mix 15 Min-1 mg-Initial | 118.75 | 0.18 | ND |
| Lubricated Blend-1 mg-Initial | 120 | 0.19 | ND |
| Core Tablets-1 mg-Initial | 120 | 0.19 | ND |
| Core Tablets-1 mg-Initial | 120 | 0.10 | ND |
| (40/75, 7 days, Open) | | | |
| Core Tablets-1 mg-Initial | 120 | 0.71 | ND |
| (60°, 7 days, Open) | | | |

Batch with Dicalcium phosphate-1—Formulation 1

| | | | |
|---|---|---|---|
| Dry Mix 15 Min-1 mg-Initial | 118.75 | 0.18 | ND |
| Lubricated Blend-1 mg-Initial | 120 | 0.18 | ND |
| Core Tablets-1 mg-Initial | 120 | 0.10 | ND |
| Core Tablets-1 mg-Initial | 120 | 1.14 | ND |
| (40/75, 7 days, Open) | | | |
| Core Tablets-1 mg-Initial | 120 | 1.24 | ND |
| (60°, 7 days, Open) | | | |

Batch with Dicalcium phosphate-2—Formulation 2

| | | | |
|---|---|---|---|
| Dry Mix 15 Min-1 mg-Initial | 118.75 | 0.17 | ND |
| Lubricated Blend-1 mg-Initial | 120 | 0.27 | ND |
| Core Tablets-1 mg-Initial | 120 | 0.26 | ND |
| Core Tablets-1 mg-Initial | 120 | 5.36 | ND |
| (40/75, 7 days, Open) | | | |
| Core Tablets-1 mg-Initial | 120 | 3.34 | ND |
| (60°, 7 days, Open) | | | |

Batch with DCP and Sodium Bicarbonate—Formulation 3

| | | | |
|---|---|---|---|
| Dry Mix 15 Min-1 mg-Initial | 118.75 | 0.23 | ND |
| Lubricated Blend-1 mg-Initial | 120 | 0.26 | ND |
| Core Tablets-1 mg-Initial | 120 | 0.30 | ND |
| Core Tablets-1 mg-Initial | 120 | 6.77 | ND |
| (40/75, 7 days, Open) | | | |
| Core Tablets-1 mg-Initial | 120 | 0.93 | ND |
| (60°, 7 days, Open) | | | |

Batch with Dicalcium phosphate-3—Formulation 4

| | | | |
|---|---|---|---|
| Dry Mix 15 Min-0.5 mg-Initial | 59.5 | 0.18 | ND |
| Lubricated Blend-0.5 mg-Initial | 60 | 0.12 | ND |
| Core Tablets-1 mg-Initial | 60 | 0.22 | ND |
| Core Tablets-1 mg-Initial | 60 | Not | Not |
| (40/75, 7 days, Open) | | available | available |
| Core Tablets-1 mg-Initial | 60 | Not | Not |
| (60°, 7 days, Open) | | available | available |

While examples of certain particular embodiments are provided herein, it will be apparent to those skilled in the art that various changes and modifications may be made. Such modifications are also intended to fall within the scope of the appended claims.

As depicted in table 2, nitrosamine impurities (specifically VC09) were found less than 9 ppm (within the daily acceptable intake limit) in all the batches.

Above examples thus demonstrate that using alkaline or basic excipients, or tartaric acid or keeping weight percent of varenicline base 0.70% or more as that of total weight of composition, are efficiently keeping the said composition stable with nitrosamine impurities within the daily acceptable intake limit.

Both the foregoing summary and the detailed description are exemplary and explanatory. They are intended to provide further details of the invention, but are not to be construed as limiting. Other objects, advantages, and novel features will be readily apparent to those skilled in the art from the detailed description of the invention.

1. A stable pharmaceutical composition of varenicline or pharmaceutically acceptable salts thereof, comprising one or more pharmaceutically acceptable excipients selected from a) at least one organic or inorganic basic excipient or b) at least one organic or inorganic acid wherein the composition contains nitrosamine impurities within daily acceptable limit.

2. The stable pharmaceutical composition according to claim 1, further comprises one or more other pharmaceutically acceptable excipients.

3. The stable pharmaceutical composition according to claim 1, wherein at least one organic or inorganic basic excipient include but are not limited to carbohydrates, meglumine, starch, cellulose, petrochemicals, povidones, mineral hydrocarbons, acrylic polymers, oleo chemicals, amines or proteins or combinations thereof.

4. The stable pharmaceutical composition according to claim 3, wherein at least one organic or inorganic basic excipient include dicalcium phosphate, sodium carbonate, sodium bicarbonate or combinations thereof.

5. The stable pharmaceutical composition according to claim 1, wherein the weak acids include but are not limited to the organic acid or inorganic acids including but not limited to tartaric, fumaric, citric or adipic acid.

6. The stable pharmaceutical composition according to claim 1, wherein the weak acid is tartaric acid and wherein the alkaline or basic excipient is selected from dicalcium phosphate or sodium bicarbonate.

7. The stable pharmaceutical composition according to claim 1, wherein the composition comprises varenicline or pharmaceutically acceptable salts thereof and one or more of diluents, lubricants, weak acids, alkaline or basic excipients, film coating agents.

8. The stable pharmaceutical composition according to claim 1, wherein the composition comprises varenicline tartrate.

9. The stable pharmaceutical composition according to claim 1, wherein the composition comprises of varenicline base whose weight percent is 0.4% to 0.7% of total weight of composition.

10. The stable pharmaceutical composition according to claim 1, wherein the weight ratio of varenicline to weak acid is 0.008:1 to 0.025:1 and wherein the weight ratio of varenicline to alkaline or basic excipient is about 1:2 to 1:90.

11. A stable pharmaceutical composition according to claim 1, comprising one or more pharmaceutically acceptable excipients selected from a) at least one organic or inorganic basic excipient or b) at least one organic or inorganic acid wherein the composition contains nitrosamine impurities within daily acceptable limit, wherein the daily acceptable limit is 9 ppm.
